(19) United States
(12) Patent Application Publication
(10) Pub. No.: US 2026/0216438 A1
(43) Pub. Date: Jul. 30, 2026

(54) VACUUM SYRINGE PLUNGER CLIP

(71) Applicant: CareFusion 303, Inc., San Diego, CA (US)

(72) Inventors: Eugene E. MASON, La Habra Heights, CA (US); Jason Andrew Wine, Brea, CA (US); Likhith Nalluri, Brea, CA (US)

(21) Appl. No.: 19/041,758

(22) Filed: Jan. 30, 2025

(51) Int. Cl. A61M 5/31; A61J 1/20; A61M 5/315

(52) U.S. Cl. CPC A61M 5/3146; A61M 5/31505; A61J 1/2096; A61M 2005/31508

(57) ABSTRACT

The priming attachment for a vacuum syringe can prevent air that is drawn out of the IV set and into the vacuum syringe from being released into the environment or back into the IV set. The priming attachment couples to the vacuum syringe and prevents the syringe plunger from reentering the syringe barrel. One embodiment of the priming attachment includes arms that fit inside the barrel and are pulled distally until they are released from the barrel. Once the arms released, the arms cannot reenter the barrel, which prevents distal movement of the plunger relative to the barrel. Another embodiment of the priming attachment includes a key that couples with a notch in a modified plunger. The modified plunger can rotate between a free position, where it can reciprocate within the barrel, and a locked position, where the notch aligns with the key and prevents translation of the modified plunger.

### TECHNICAL FIELD

The present application relates generally to a system and method for priming an intravenous line and, more particularly, vacuum syringes and vacuum syringe attachments.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, a syringe. Certain configurations of IV sets may have extended lengths of tubing, for example, in excess of 6 feet. It is very common for air bubbles to get trapped in the IV set and the tubing.

Therefore, before connecting an IV line to an infusion container containing a medical fluid, the IV line needs to be primed to remove the air bubbles. This can be done by using a vacuum syringe to remove air from the infusion container. However, in cases where the medical fluid is a hazardous drug (e.g., a chemotherapeutic drug), health care providers administering the hazardous drugs can be at risk of exposure to these medications, which presents a major hazard in the health care environment. Unintentional chemotherapy exposure can affect the nervous system, impair the reproductive system, and bring an increased risk of developing blood cancers in the future.

### SUMMARY

An IV set that contains medication to be delivered to a patient must first be primed to remove the air from the IV set. Where the medication is hazardous to caregivers (e.g., chemotherapeutic medications), priming into a waste disposal container can expose the caregiver to the hazardous drugs. Even priming with a vented cap can be dangerous and expose the caregiver to contaminated air. In order to reduce the risk of health care providers being exposed to hazardous chemicals, a tool for priming hazardous medications in a closed environment is desirable. A vacuum syringe can be coupled to an IV set (e.g., an extension tube, a fluid connector, or the IV bag itself) to draw air out of the IV set in order to prime the IV set before the medication is delivered to the patient. A priming attachment can be clipped onto the vacuum syringe to prevent the contaminated air from leaking out of the vacuum syringe and exposing caregivers to dangerous chemicals.

Some embodiments of the present disclosure are directed to a priming attachment for a vacuum syringe, the priming attachment comprising a proximal cap comprising one or more engagement features configured to couple to a plunger flange of the vacuum syringe and stoppers extending distally from at least one of the one or more engagement features. Each stopper comprises an alignment section that protrudes from a respective engagement feature and extends radially inward, the alignment section being configured to align the stopper with a recess in a plunger of the vacuum syringe; an arm that extends distally from the alignment section, the arm being configured to extend along the recess and fit within a barrel of the vacuum syringe and bear radially outward against the barrel; and a lip that protrudes from a distal portion of the arm, the lip being configured to restrict the plunger from reentering the barrel after the plunger and the stoppers are withdrawn from the barrel.

In some embodiments, the proximal cap is flat and circular.

In some embodiments, the one or more engagement features are positioned on an outer perimeter of the proximal cap. Optionally, the one or more engagement features are configured to snap onto the plunger flange. Optionally, each engagement feature of the one or more engagement features is at least about 90° away from an adjacent engagement feature.

In some embodiments, an angle between the alignment section and the arm is between about 150° and 160°.

In some embodiments, the arm of each stopper further comprises at least one sub-arm. Each sub-arm extends distally from and radially away from the arm and has a corresponding lip that protrudes from a distal portion of a respective additional arm. Moreover, each sub-arm is configured to be compressed onto the arm to fit within the barrel of the vacuum syringe and bear radially outward against the barrel. Additionally, each corresponding lip is configured to restrict the plunger from reentering the barrel. Optionally, each sub-arm is positioned at a different location along the respective arm to accommodate a different volume of fluid inside the barrel. Optionally, each sub-arm extends distally from and radially away from the arm at an angle between about 155° and 165°.

In some embodiments, the lip comprises a sloped edge and a flat end, the flat end being configured to abut a proximal end of the barrel when the sloped edge is withdrawn from the barrel in order to restrict the plunger from reentering the barrel after the plunger and the stoppers are withdrawn from the barrel. Optionally, the flat end is perpendicular to the arm.

Some embodiments of the present disclosure are directed to a priming attachment for a vacuum syringe that comprises a proximal cap comprising one or more engagement features configured to couple to a plunger flange of the vacuum syringe and one or more stoppers comprising one or more arms, each of the one or more arms being configured to extend along a recess in a plunger of the vacuum syringe and bear radially outward against a barrel of the vacuum syringe while the plunger and the one or more stoppers are inside the barrel. Moreover, a distal portion of each arm comprises a lip configured to restrict the plunger from reentering the barrel after the plunger and the stoppers are withdrawn from the barrel.

In some embodiments, each stopper of the one or more stoppers comprises three arms. Optionally, each stopper of the one or more stoppers extends distally from an engagement feature of the one or more engagement features.

In some embodiments, the lip comprises a sloped edge and a flat end, the flat end being configured to abut a proximal end of the barrel when the sloped edge is withdrawn from the barrel in order to restrict the plunger from reentering the barrel after the plunger and the stoppers are withdrawn from the barrel.

Other embodiments of the present disclosure are directed to a priming system for a vacuum syringe, the priming system comprising a priming attachment configured to be coupled to a barrel flange of the vacuum syringe and comprising one or more keys and a plunger comprising one or more notches, the plunger being configured to rotate relative to the priming attachment between a free position in which the plunger is configured to reciprocate within the barrel and a locked position in which the plunger cannot reciprocate within the barrel. In the free position, the one or more notches are not aligned with any of the one or more keys, and in the locked position, at least one of the one or more notches aligns with at least one of the one or more keys.

In some embodiments, the priming attachment is configured to at least partially cover the barrel flange and surround a proximal barrel opening of the vacuum syringe, and the one or more keys protrude from the priming attachment and are configured to extend partially over the proximal opening. In some embodiments, the priming attachment comprises one or more engagement features configured to snap onto the barrel flange.

In some embodiments, each of the one or more keys protrudes radially inward, is at least as deep as each of the one or more notches and is wider than each of the one or more notches.

In some embodiments, the one or more notches are located at different positions along a length of the plunger, wherein the different positions along the length of the plunger are configured to correspond with different volumes of fluid inside a barrel of the vacuum syringe.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates a vacuum syringe being used to remove air from the IV set, in accordance with some embodiments disclosed herein.

FIG. 2 illustrates a first embodiment of a priming attachment coupled to the vacuum syringe, in accordance with some embodiments disclosed herein.

FIGS. 3A and 3B illustrate perspective and front views of the first embodiment of the priming attachment, in accordance with some embodiments disclosed herein.

FIG. 4 illustrates a second embodiment of the priming attachment coupled to the vacuum syringe, in accordance with some embodiments disclosed herein.

FIGS. 5A and 5B illustrate perspective views of the second embodiment of the priming attachment, in accordance with some embodiments disclosed herein.

### DETAILED DESCRIPTION

In the following detailed description, numerous specific details are set forth to provide a full understanding of the subject technology. The subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

Priming an IV set is an essential step that precedes delivering chemotherapeutic medication to a patient intravenously. Vacuum syringes may be coupled to an IV set to remove air from an IV bag. A caregiver can proximally pull on the plunger of the vacuum syringe to remove the air from the IV bag and draw the air into the barrel of the plunger. However, using a typical syringe to prime an IV set has drawbacks. For example, it would be easy for a caregiver to accidentally release contaminated air into the environment by accidentally pushing on the plunger. It is also possible for the vacuum formed inside the barrel of the syringe to apply a distal force to the plunger that pulls the plunger distally and releases contaminated air into the environment. The following devices and methods provide design modifications to overcome the foregoing issues.

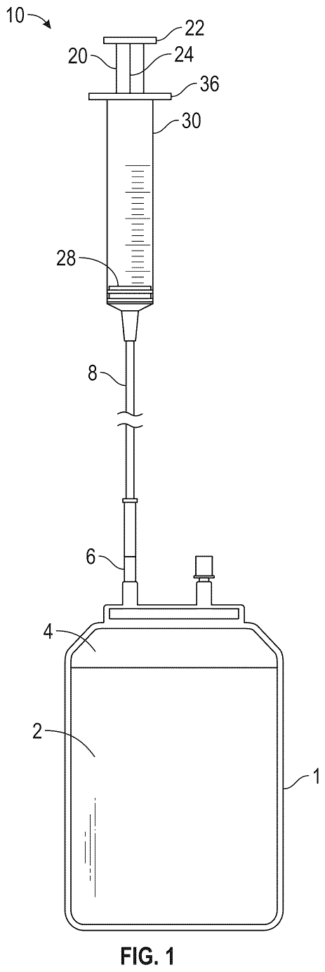

Referring now to the figures, FIG. 1 illustrates a vacuum syringe being used to remove air from the IV set, in accordance with some embodiments disclosed herein. In FIG. 1, the IV bag 1 contains a medication 2. Before a caregiver can deliver the medication 2 to a patient, the air 4 must be removed from the IV bag 1. This can be done by coupling a vacuum syringe 10 to a port 6 in the IV bag 1 via an IV line or extension tube 8 and drawing the air 4 from the IV bag 1 into the barrel 30 of the vacuum syringe 10. This can also be done by coupling the vacuum syringe 10 directly to the port 6 without the IV line or extension tube 8.

The vacuum syringe 10 is coupled to the IV bag 1 when the vacuum syringe 10 is in an empty configuration. In this configuration, most of the plunger 20 of the vacuum syringe 10 is within the barrel 30. Additionally, the seal 28 of the plunger 20 can be at the distalmost end of the barrel 30 when the vacuum syringe 10 is in the empty configuration. After the vacuum syringe 10 is coupled to the IV bag 1, the caregiver can withdraw air 4 from the IV bag 1 and into the barrel 30 of the vacuum syringe by pulling distally on the plunger flange 22 of the plunger 20. In order to ensure that all of the air 4 is removed from the IV bag 1, the caregiver might also withdraw some of the medication 2 into the barrel 30 of the vacuum syringe 10.

In cases where the medication 2 is hazardous, such as with chemotherapeutic medication, the caregiver should avoid contact with both the air 4 and the medication 2. This can be accomplished with a priming attachment, which is described in greater detail below.

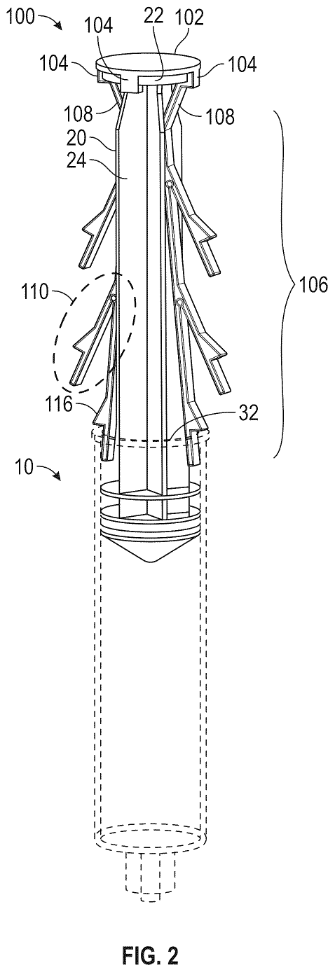

FIG. 2 illustrates a first embodiment of a priming attachment coupled to the vacuum syringe, in accordance with some embodiments disclosed herein. The priming attachment 100 can be snapped, clipped, or otherwise coupled with the plunger 20 of the vacuum syringe 10. Moreover, the priming attachment 100 is designed to attach to any standard syringe.

The priming attachment 100 is configured to hold the plunger 20 in place relative to the barrel 30 after the air 4 and/or the medication 2 has been drawn into the barrel 30. In particular, the priming attachment 100 prevents the plunger 20 from reentering the barrel 30 and releasing the air 4 and/or the medication 2 from the vacuum syringe 10. Because the priming attachment 100 holds the plunger 20 in place, the caregiver can put down the vacuum syringe 10 without worrying about exposure to hazardous chemicals. Thus, the priming attachment 100 makes priming the IV bag 1 more convenient for the caregiver, as compared to traditional methods.

The priming attachment 100 has a proximal cap 102 that is configured to be attached to the plunger flange 22 of the plunger 20. The proximal cap 102 is generally flat and has the same shape as the plunger flange 22 so that the priming attachment 100 does not inconveniently increase the size of the vacuum syringe 100. In FIG. 2, the proximal cap 102 is a flat circle that is flush against the plunger flange 22, which is also circular. In some embodiments, the proximal cap 102 is an ellipse. Other shapes are also possible.

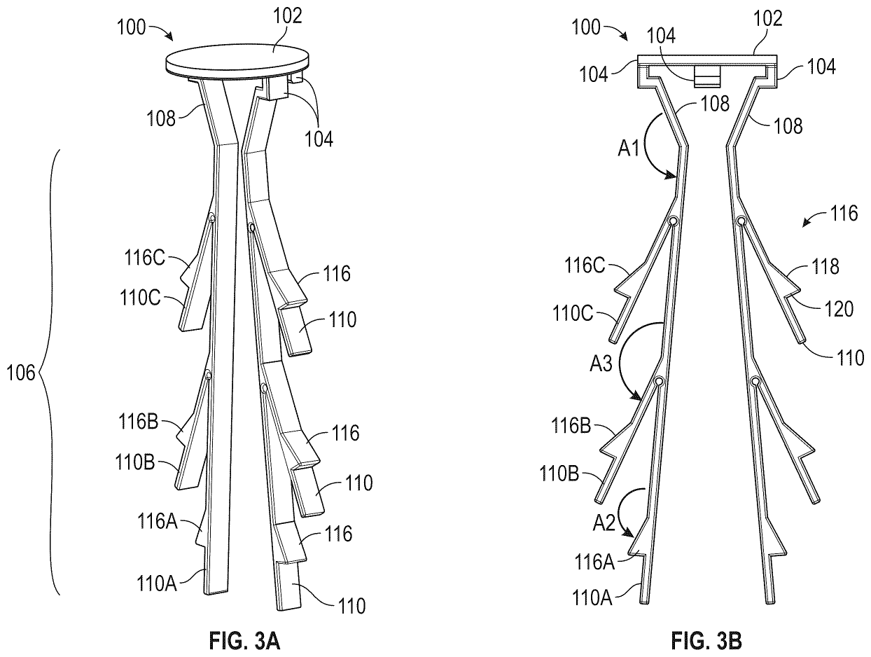

The priming attachment 100 has one or more engagement features 104 positioned along an outer perimeter of the proximal cap 102. The engagement features 104 are configured to snap fit the proximal cap 102 to the plunger flange 22. In the embodiment shown in FIG. 2, each engagement feature 104 is an L-shaped shelf that can clip onto an outer edge of the plunger flange 22. Other shapes for the engagement feature 104 are also possible. In the embodiment shown in FIG. 2, the proximal cap 102 only has three engagement features 104. This is shown in FIGS. 3A and 3B, which are discussed in greater detail below. In some embodiments, the proximal cap 102 only has one engagement feature 104. In other embodiments, the proximal cap 102 has two engagement features 104. Optionally, the proximal cap 102 has more than three engagement features 104.

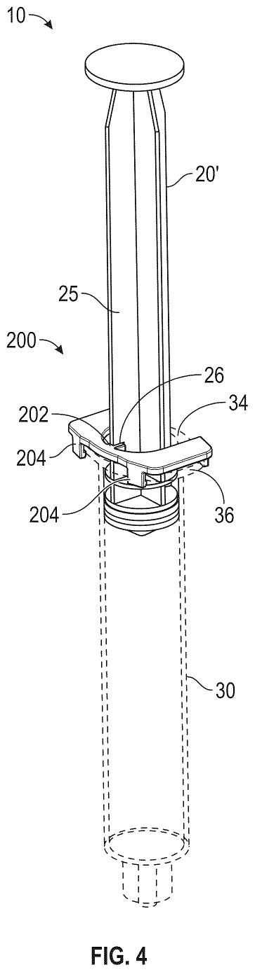

The engagement features 104 are positioned on the proximal cap 102 such that the engagement features 104 do not interfere with the plunger 20 of the vacuum syringe. The plunger 20 shown in the present disclosure has a plus-shaped cross-section made up of four walls and four recesses 24. This can be seen in FIGS. 1 and 4 as well as in FIG. 2. In FIG. 2, each of the engagement features 104 is at least 90° away from a neighboring engagement feature 104 so that each engagement feature 104 is aligned with a recess 24 of the plunger 20.

At least one of the engagement features 104 is part of a stopper 106. In FIG. 2, two of the engagement features 104 extend into stoppers 106. The stoppers 106 extend distally away from the proximal cap 102 and are configured to fit inside the recesses 24 of the plunger 20. Each stopper 106 has an alignment section 108 that extends from the engagement feature 104 radially inward. That is, when the priming attachment 100 is coupled to the plunger 20 of the vacuum syringe 100, each alignment section 108 extends from the engagement feature 104 towards a recess 24 of the plunger 20. Consequently, each alignment section 108 facilitates alignment and storage of each stopper 106 within a respective recess 24 of the plunger 20. When the plunger 20 is inside the barrel 30, each stopper 106 is stored in a recess 24 of the plunger 20.

Each stopper 106 also has at least one arm 110. Each arm 110 has a lip 116 that is configured to prevent the plunger 20 from reentering the barrel 30 after the plunger 20 and the stoppers 106 are withdrawn from the barrel 30. Specifically, after the lip 116 is outside of the barrel 30, the distal end of the lip 116 will abut the proximal end 32 of the barrel 30 and resist distal force applied to the plunger. The arms 110 and the lips 116 are described in greater detail below with respect to FIGS. 3A and 3B.

FIGS. 3A and 3B illustrate perspective and front views of the first embodiment of the priming attachment, in accordance with some embodiments disclosed herein. With reference to FIG. 3A, a first arm 110A is a long, flat rectangular panel (e.g., a vertical cantilever that is supported at its proximal end by the distal end of the alignment section 108) that extends distally from the distal end of the alignment section 108. As shown in FIG. 3B, the first arm 110 extends from the alignment section 108 at an angle A1 of approximately 152.5°. In some embodiments, the angle A1 between the first arm 110 and the alignment section 108 is between about 147° and 157°. In other embodiments, the angle A1 is between about 142° and 162°.

As described above and shown in FIG. 3A, the first arm 110A has a lip 116A positioned at a distal portion of the first arm 110A. Each lip 116 protrudes from a distal portion of a respective arm 110 and comprises a triangular shape made up of a sloped edge 118 and a flat end 120 (shown in FIG. 3B). The sloped edge 118 protrudes away from the arm 110 at an angle A2 of approximately 157°. In some embodiments, the angle A2 between the sloped edge 118 and the arm 110 is between about 152° and 162°. In other embodiments, the angle A2 is between about 147° and 167°. The flat end 120 is generally transverse to the arm 110.

The first arm 110A is configured to be squeezed into a recess 24 of the plunger 20, as shown in FIG. 2. When the plunger 20 is inside the barrel 30 of the vacuum syringe 10, the first arm 110A (specifically, the lip 116A) bears against the inner walls of the barrel 30 because the first arm 110A is biased radially outward. Because the first arm 110A is pressed against the inner walls of the barrel 30, when the caregiver withdraws the plunger 20 (and, thus, the priming attachment 100 and the stopper 106) from the barrel 30, the proximal end 32 of the barrel 30 slides against the sloped edge 118 of the lip 116A until the lip 116A is fully withdrawn from the barrel 30. At that point, if the caregiver stops proximally pulling on the plunger 20, the proximal end 32 of the barrel 30 is adjacent to the flat end 120 of the lip 116A. As described above with respect to FIG. 2, in this position, if the caregiver pushes distally on the plunger 20 or if the vacuum inside the barrel 30 applies a distal force on the plunger 20, the priming attachment 100 will resist movement of the plunger because the flat end 120 is pressing against the proximal end 32 of the barrel 30.

In some embodiments, a stopper 106 can have additional arms 110. In the embodiment shown, the additional arms 110 are smaller than the first arm 110A and can be referred to as sub-arms. With reference to FIG. 3A, the stopper 106 has second and third arms 110B, 110C (or sub-arms 110B, 110C) that protrude from the first arm 110A. Optionally, the stopper 106 only has two arms 110. Optionally, the stopper 106 has more than three arms 110. As shown, the second and third arms 110B, 110C are flat rectangular panels that are shorter than the first arm 110A. In some embodiments, the second and third arms 110B, 110C are about one third the length of the first arm 110A. In other embodiments, the second arm 110B has a different length than the third arm 110C.

Each of the second and third arms 110B, 110C extend distally from and radially away from the first arm 110A. In particular, the second and third arms 110B, 110C each extend away from the first arm 110A at an angle A3 of approximately 159.5°. In some embodiments, the angle A3 between each of the second and third arms 110B, 110C and the first arm 110 is between about 154° and 164°. In other embodiments, the angle A3 is between about 149° and 169°.

Each of the second and third arms 110B, 110C has a lip 116B, 116C positioned at a distal portion of the second and third arms 110B, 110C, respectively. As described above, each lip 116 (which includes 116A, 116B, and 116C) comprises a triangular protrusion with a sloped edge 118 that generally faces the proximal direction and a flat edge 120 that generally faces the distal direction. The sloped edge 118 forms an angle A2 with the arm 110, and the flat edge 120 is approximately perpendicular to the arm 110.

Like the first arm 110A, each of the second and third arms 110B, 110C can be pushed radially inwards to fit inside a recess 24 of the plunger 20 when the plunger 20 is inside the barrel 30. Similarly, when the second and third arms 110B, 110C are inside the barrel 30, the second and third arms 110B, 110C are biased radially outward and the lips 116B, 116C press against the inner wall of the barrel 30. Because the second and third arms 110B, 110C bear radially outward against the barrel 30, when, for example, the second arm 110B is withdrawn from the barrel 30, the proximal end 32 of the barrel 30 slides against the sloped edge 118 of the lip 116B until the lip 116B is fully withdrawn from the barrel. At that point, if the caregiver tries to push the plunger 20 distally, or if the vacuum formed inside the barrel 30 applies a distal suction force to the plunger 20, the flat end 120 of the lip 116B will prevent the plunger 20 from sliding back into the barrel 30. The same is true for the third arm 110C and the corresponding lip 110C.

Additionally, each of the second and third arms 110B, 110C is positioned at a different location along the length of the first arm 110A. For example, the second arm 110B can be approximately halfway down the first arm 110A while the third arm 110C is near the proximal end of the first arm 110A. Other positions are also possible. These different positions are configured to accommodate different volumes of air and medication (e.g., air 4 and medication 2 in FIG. 1) inside the barrel 30 of the vacuum syringe 10. In other words, each of the different arms 110 can be used to resist reentrance of the plunger 20 back into the barrel 30 when there are different volumes of fluid (e.g., air 4 and medication 2) inside the barrel 30 of the vacuum syringe 10.

The present application also discloses methods for using the vacuum syringe 10 with the priming attachment 100. These steps are described with reference to FIGS. 1-3B of the present application. First, the priming attachment 100 is clipped onto the vacuum syringe 10. In particular, the proximal cap 102 of the priming attachment is snapped onto the plunger flange 22 of the vacuum syringe's 10 plunger 20. The plunger 20 is distally advanced into the barrel 30 with the stoppers 106 of the priming attachment being compressed to fit inside the recesses 24 of the plunger 20. The plunger 20 is distally advanced until the seal 28 is at a distalmost end of the barrel 30.

Next, the vacuum syringe 10 with the priming attachment 100 is coupled to an IV bag 1 so that the IV bag 1 can be primed. The plunger 20 is pulled proximally to draw the air 4 (and some of the medicine 2) out of the IV bag 1 and into the barrel 30 of the vacuum syringe 10. As the plunger 20 is pulled proximally, the lips 116 on a first set of arms 110 bear against the inner surface of the barrel 30. (Each “set” of arms 110 includes all of the arms 110 that are the same distance away from the proximal cap 102. Here, the first set of arms 110 includes all of the arms 110 that are the furthest away from the proximal cap 102.) When the distal end of the sloped edges 118 of the lips 116 of the first set of arms 110 pass the threshold of the proximal end 32 of the barrel 30, the lips 116 of the first set of arms 110 pop out of barrel 30, and the portions of the arms 110 that are distal to the lips 116 bear against the inner surface of the barrel 30. The flat ends 120 of the lips 116 prevent the plunger from sliding back into the barrel 30, which prevents the air 4 and the medicine 2 from being released from the vacuum inside the barrel 30.

Where there are multiple sets of arms 110 on each stopper 106, the plunger 20 can continue to be pulled proximally to remove the next set of arms 110 from the barrel 30 and draw a greater volume of air 4 and medication 2 from the IV bag 1 into the barrel 30. When the plunger 20 is withdrawn proximally to remove the next set of arms 110 from the barrel 30, the portions of the arms 110 that are distal to the lips 116 pop out of the barrel 30. As described above with respect to the first set of arms 110, the flat ends 120 of the lips 116 restrict the plunger 20 from reentering the barrel 30 at this point.

Each set of arms 110 corresponds to a different volume of fluid (e.g., air 4 and medication 2) inside the barrel 30 of the vacuum syringe 10. As a result, the caretaker using the vacuum syringe 10 and the priming attachment 100 can identify the volume of fluid inside the barrel 30 by listening to the sound of the lips 116 popping out of the barrel 30 or feeling for the tactile sensation of the lips 116 being withdrawn from the barrel.

FIG. 4 illustrates a second embodiment of the priming attachment coupled to the vacuum syringe, in accordance with some embodiments disclosed herein. The priming attachment 200 can be snapped, clipped, or otherwise coupled to the barrel flange 36 of the vacuum syringe 10. The priming attachment 200 is designed to be used with a modified plunger 20′. The priming attachment 200 and the modified 20′ are designed to be used with any standard barrel 30 of a vacuum syringe 10.

The vacuum syringe 10 has a modified plunger 20′. The modified plunger 20′ has a plus-shaped cross-section made up of four walls 25 and four recesses. As shown in FIG. 4, the modified plunger 20′ can have a notch 26 in one of the walls 25. The notch 26 in the wall 25 of the modified plunger 20′ can be removably coupled to a key 202 in the priming attachment 200. When the notch 26 is not coupled to the key 202, the modified plunger 20′ and the priming attachment 200 are in a free position. In the free position, the modified plunger 20′ can reciprocate within the barrel 30. When the notch 26 is coupled to the key 202, the modified plunger 20′ and the priming attachment 200 are in a locked position. In the locked position, the modified plunger 20′ cannot reciprocate within the barrel 30 of the vacuum syringe 10.

The modified plunger 20′ can be rotated relative to the priming attachment 200 between the free position and the locked position by misaligning and aligning the notch 26 with the key 202. When the caregiver is priming an IV set (e.g., IV bag 1, shown in FIG. 1), the caregiver can rotate the modified plunger 20′ into the free position to withdraw the modified plunger 20′ and draw air from the IV set. When the caregiver is done removing the air from the IV set (or needs to pause while priming the IV set), the caregiver can rotate the modified plunger 20′ into the locked position to prevent the modified plunger 20′ from reentering the barrel 30 of the vacuum syringe 10. Thus, the combination of the modified plunger 20′ and the priming attachment can serve the same purpose as the priming attachment 100, described above.

In some embodiments, the modified plunger 20′ has multiple notches 26. The notches 26 can be positioned in different walls 25 at the same distance away from the plunger flange (i.e., at the same “level”). The notches 26 can also be positioned at different distances away from the plunger flange (i.e., at different levels) in the same wall 25 or in different walls 25. The notches 26 being positioned at different distances away from the plunger flange would correspond to different volumes of fluid inside the barrel.

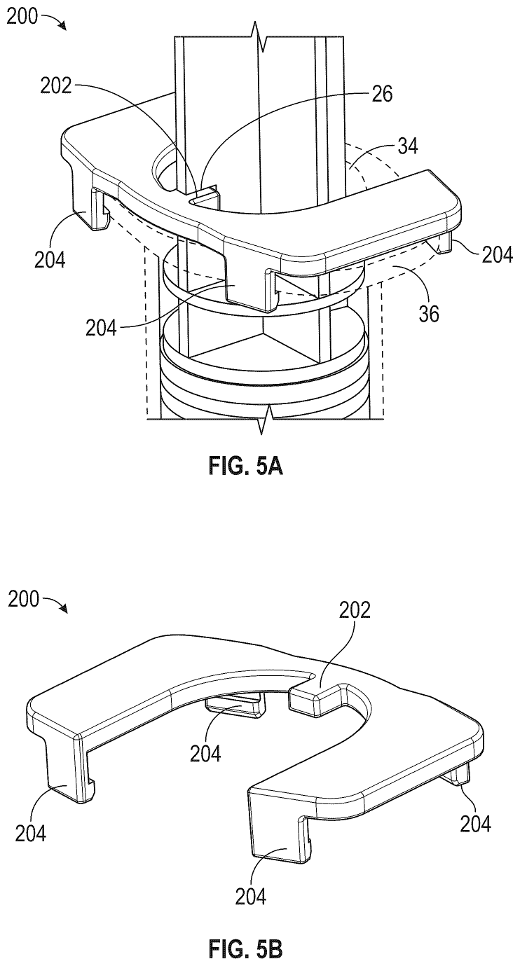

FIGS. 5A and 5B illustrate perspective views of the second embodiment of the priming attachment, in accordance with some embodiments disclosed herein. As shown in FIG. 5A, the priming attachment 200 clips onto the barrel flange 36 of the vacuum syringe and covers most of the barrel flange 36. The priming attachment 200 in FIGS. 5A and 5B is generally U-shaped such that the priming attachment 200 surrounds the proximal opening 34 of the vacuum syringe barrel so that the modified plunger (i.e., the modified plunger 20′ shown in FIG. 4) can enter the barrel through the proximal opening 34. Optionally, the priming attachment 200 can fully cover the barrel flange 36 and have a hole in the middle that exposes the proximal opening 34 of the vacuum barrel syringe.

The priming attachment 200 has a key 202 that couples with the notch 26 in the modified syringe. In FIGS. 5A and 5B, the key 202 is a generally rectangular protrusion that extends radially inward from the base of the U-shaped opening of the priming attachment 200. The key 202 is slightly wider than the notch 26, slightly thinner than the notch 26, and almost as deep as the notch 26, such that the key 202 can slide through the notch 26 as the modified plunger is rotated relative to the vacuum syringe barrel and catch the notch 26 to prevent movement of the modified plunger in and out of the barrel when the modified syringe is in the locked position. Other shapes for the key 202 are also possible. For example, the key 202 can be semi-circular or trapezoidal. A wider key 202 would make rotating the modified syringe into the locked position easier than a narrower key 202, which would be more difficult to align with the notch 26.

In some embodiments, the priming attachment 200 has multiple keys 202. Optionally, the multiple keys 202 can each be configured to couple with one notch 26 at each level of notches 26 along the modified plunger. This configuration would make it easier for the caregiver to rotate the modified plunger into the locked position because there are more keys 202 available to align with any given notch 26. Optionally, the multiple keys 202 can each be configured to align with a different notch 26 in different walls 25 at a singular level of notches 26 along the modified plunger. This would add increased resistance to the distal movement of the modified plunger back into the barrel. Each of the keys 202 in the priming attachment would align with a notch 26 in the modified plunger. For example, where the modified plunger is designed such that the walls 25 meet to form a 90°, the keys 202 are 90° away from adjacent keys 202, such that each key 202 aligns with a notch 26.

The priming attachment 200 also includes engagement features 204 that protrude distally from the outer perimeter of the priming attachment 200. The engagement features 204 are configured to clip onto the barrel flange 36 and secure the priming attachment to the vacuum syringe. The engagement features 204 are L-shaped shelves that are long enough to clip onto the barrel flange 36 of a standard syringe.

The present application also discloses methods for using the vacuum syringe 10 with the priming attachment 200. These steps are described with reference to FIGS. 1 and 4-5B of the present application. First, the key 202 (or keys 202) of the priming attachment 200 is aligned with the notch 26 (or notches 26) of the modified plunger 20′. Next, the distal end of the modified plunger 20′ (e.g., the seal 28) is slid into the barrel 30 of the vacuum syringe 10, and the priming attachment 200 is clipped onto the barrel flange 36 of the barrel 30 via the engagement features 204 of the priming attachment 200. The modified plunger 20′ is rotated into the free position (i.e., the notch 26 is rotated out of the key 202), and the modified plunger 20′ is distally advanced inside the barrel 30 until the seal 28 is at the distalmost end of the barrel 30.

Next, the vacuum syringe 10 with the priming attachment 200 and the modified plunger 20′ is coupled to an IV bag 1 so that the IV bag 1 can be primed. The modified plunger 20′, which is still in the free position, is pulled proximally to draw the air 4 (and some of the medicine 2) out of the IV bag 1 and into the barrel 30 of the vacuum syringe 10. When all of the air 4 has been withdrawn from the IV bag 1 (or the caretaker needs to pause priming), the modified plunger 20′ can be rotated such that the notch 26 on the modified plunger 20′ aligns with a key 202 in the priming attachment 200. The key 202 of the priming attachment 200 restricts movement of the modified plunger 20′ such that the modified plunger 20′ cannot reciprocate within the barrel 30 of the vacuum syringe 10. This prevents the fluid inside the barrel 30 (i.e., air 4 and medication 2) from being released from the vacuum chamber inside the barrel 30 of the vacuum syringe 10 and contaminating the surrounding environment.

Where there are multiple levels of notches 26 on the modified plunger 20′, the modified plunger 20′ can be rotated back into the free position and continue to be pulled proximally to draw a greater volume of air 4 and medication 2 out of the IV bag 1. When the priming is complete, the modified plunger 20′ can be rotated back into the locked position, such that the notch 26 at the next level couples with the key 202 to once again restrict the modified plunger 20′ from reentering the barrel 30.

Each level of notches 26 corresponds to a different volume of fluid inside the barrel 30 of the vacuum syringe 10. As a result, the caretaker using the vacuum syringe 10 with the priming attachment 200 and the modified plunger 20′ can identify the volume of fluid inside the barrel 30 by visually identifying which notch 26 is coupled to the key 202.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 9, or clause 16. The other clauses can be presented in a similar manner.

Clause 1. A priming attachment for a vacuum syringe, the priming attachment comprising (a) a proximal cap comprising one or more engagement features configured to couple to a plunger flange of the vacuum syringe and (b) stoppers extending distally from at least one of the one or more engagement features, wherein each stopper comprises: an alignment section that protrudes from a respective engagement feature and extends radially inward, the alignment section being configured to align the stopper with a recess in a plunger of the vacuum syringe; an arm that extends distally from the alignment section, the arm being configured to extend along the recess and fit within a barrel of the vacuum syringe and bear radially outward against the barrel; and a lip that protrudes from a distal portion of the arm, the lip being configured to restrict the plunger from reentering the barrel after the plunger and the stoppers are withdrawn from the barrel.

Clause 2. The priming attachment of Clause 1, wherein the proximal cap is flat and circular.

Clause 3. The priming attachment of Clause 1, wherein the one or more engagement features are positioned on an outer perimeter of the proximal cap.

Clause 4. The priming attachment of Clause 1, wherein the one or more engagement features are configured to snap onto the plunger flange.

Clause 5. The priming attachment of Clause 1, wherein each engagement feature of the one or more engagement features is at least about 90° away from an adjacent engagement feature.

Clause 6. The priming attachment of Clause 1, wherein an angle between the alignment section and the arm is between about 150° and 160°.

Clause 7. The priming attachment of Clause 1, wherein the arm of each stopper further comprises at least one sub-arm, wherein each sub-arm extends distally from and radially away from the arm and has a corresponding lip that protrudes from a distal portion of a respective additional arm, wherein each sub-arm is configured to be compressed onto the arm to fit within the barrel of the vacuum syringe, wherein each sub-arm is configured to bear radially outward against the barrel, and wherein each corresponding lip is configured to restrict the plunger from reentering the barrel.

Clause 8. The priming attachment of Clause 7, wherein each sub-arm is positioned at a different location along the respective arm to accommodate a different volume of fluid inside the barrel.

Clause 9. The priming attachment of Clause 7, wherein each sub-arm extends distally from and radially away from the arm at an angle between about 155° and 165°.

Clause 10. The priming attachment of Clause 1, wherein the lip comprises a sloped edge and a flat end, the flat end being configured to abut a proximal end of the barrel when the sloped edge is withdrawn from the barrel in order to restrict the plunger from reentering the barrel after the plunger and the stoppers are withdrawn from the barrel.

Clause 11. The priming attachment of Clause 10, wherein the flat end is perpendicular to the arm.

Clause 12. A priming attachment for a vacuum syringe, the priming attachment comprising:
a proximal cap comprising one or more engagement features configured to couple to a plunger flange of the vacuum syringe; and one or more stoppers comprising one or more arms, each of the one or more arms being configured to extend along a recess in a plunger of the vacuum syringe and bear radially outward against a barrel of the vacuum syringe while the plunger and the one or more stoppers are inside the barrel, wherein a distal portion of each arm comprises a lip configured to restrict the plunger from reentering the barrel after the plunger and the stoppers are withdrawn from the barrel.

Clause 13. The priming attachment of Clause 12, wherein each stopper of the one or more stoppers comprises three arms.

Clause 14. The priming attachment of Clause 12, wherein each stopper of the one or more stoppers extends distally from an engagement feature of the one or more engagement features.

Clause 15. The priming attachment of Clause 12, wherein the lip comprises a sloped edge and a flat end, the flat end being configured to abut a proximal end of the barrel when the sloped edge is withdrawn from the barrel in order to restrict the plunger from reentering the barrel after the plunger and the stoppers are withdrawn from the barrel.

Clause 16. A priming system for a vacuum syringe, the priming system comprising:
a priming attachment configured to be coupled to a barrel flange of the vacuum syringe and comprising one or more keys; and a plunger comprising one or more notches, the plunger being configured to rotate relative to the priming attachment between a free position in which the plunger is configured to reciprocate within the barrel and a locked position in which the plunger cannot reciprocate within the barrel, wherein in the free position, the one or more notches are not aligned with any of the one or more keys, wherein in the locked position, at least one of the one or more notches aligns with at least one of the one or more keys.

Clause 17. The priming system of Clause 16, wherein the priming attachment is configured to at least partially cover the barrel flange and surround a proximal barrel opening of the vacuum syringe, and the one or more keys protrude from the priming attachment and are configured to extend partially over the proximal opening.

Clause 18. The priming system of Clause 16, wherein the priming attachment comprises one or more engagement features configured to snap onto the barrel flange.

Clause 19. The priming system of Clause 16, wherein each of the one or more keys protrudes radially inward, is at least as deep as each of the one or more notches, and is wider than each of the one or more notches.

Clause 20. The priming system of Clause 16, wherein the one or more notches are located at different positions along a length of the plunger, wherein the different positions along the length of the plunger are configured to correspond with different volumes of fluid inside a barrel of the vacuum syringe.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear,” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A priming attachment for a vacuum syringe, the priming attachment comprising (a) a proximal cap comprising one or more engagement features configured to couple to a plunger flange of the vacuum syringe and (b) stoppers extending distally from at least one of the one or more engagement features, wherein each stopper comprises:
an alignment section that protrudes from a respective engagement feature and extends radially inward, the alignment section being configured to align the stopper with a recess in a plunger of the vacuum syringe;
an arm that extends distally from the alignment section, the arm being configured to extend along the recess and fit within a barrel of the vacuum syringe and bear radially outward against the barrel; and
a lip that protrudes from a distal portion of the arm, the lip being configured to restrict the plunger from reentering the barrel after the plunger and the stoppers are withdrawn from the barrel.

2. The priming attachment of claim 1, wherein the proximal cap is flat and circular.

3. The priming attachment of claim 1, wherein the one or more engagement features are positioned on an outer perimeter of the proximal cap.

4. The priming attachment of claim 1, wherein the one or more engagement features are configured to snap onto the plunger flange.

5. The priming attachment of claim 1, wherein each engagement feature of the one or more engagement features is at least about 90° away from an adjacent engagement feature.

6. The priming attachment of claim 1, wherein an angle between the alignment section and the arm is between about 150° and 160°.

7. The priming attachment of claim 1, wherein the arm of each stopper further comprises at least one sub-arm,
wherein each sub-arm extends distally from and radially away from the arm and has a corresponding lip that protrudes from a distal portion of a respective additional arm,
wherein each sub-arm is configured to be compressed onto the arm to fit within the barrel of the vacuum syringe,
wherein each sub-arm is configured to bear radially outward against the barrel, and
wherein each corresponding lip is configured to restrict the plunger from reentering the barrel.

8. The priming attachment of claim 7, wherein each sub-arm is positioned at a different location along the respective arm to accommodate a different volume of fluid inside the barrel.

9. The priming attachment of claim 7, wherein each sub-arm extends distally from and radially away from the arm at an angle between about 155° and 165°.

10. The priming attachment of claim 1, wherein the lip comprises a sloped edge and a flat end, the flat end being configured to abut a proximal end of the barrel when the sloped edge is withdrawn from the barrel in order to restrict the plunger from reentering the barrel after the plunger and the stoppers are withdrawn from the barrel.

11. The priming attachment of claim 10, wherein the flat end is perpendicular to the arm.

12. A priming attachment for a vacuum syringe, the priming attachment comprising:
a proximal cap comprising one or more engagement features configured to couple to a plunger flange of the vacuum syringe; and
one or more stoppers comprising one or more arms, each of the one or more arms being configured to extend along a recess in a plunger of the vacuum syringe and bear radially outward against a barrel of the vacuum syringe while the plunger and the one or more stoppers are inside the barrel,
wherein a distal portion of each arm comprises a lip configured to restrict the plunger from reentering the barrel after the plunger and the stoppers are withdrawn from the barrel.

13. The priming attachment of claim 12, wherein each stopper of the one or more stoppers comprises three arms.

14. The priming attachment of claim 12, wherein each stopper of the one or more stoppers extends distally from an engagement feature of the one or more engagement features.

15. The priming attachment of claim 12, wherein the lip comprises a sloped edge and a flat end, the flat end being configured to abut a proximal end of the barrel when the sloped edge is withdrawn from the barrel in order to restrict the plunger from reentering the barrel after the plunger and the stoppers are withdrawn from the barrel.

16. A priming system for a vacuum syringe, the priming system comprising:
a priming attachment configured to be coupled to a barrel flange of the vacuum syringe and comprising one or more keys; and
a plunger comprising one or more notches, the plunger being configured to rotate relative to the priming attachment between a free position in which the plunger is configured to reciprocate within the barrel and a locked position in which the plunger cannot reciprocate within the barrel,
wherein in the free position, the one or more notches are not aligned with any of the one or more keys,
wherein in the locked position, at least one of the one or more notches aligns with at least one of the one or more keys.

17. The priming system of claim 16, wherein the priming attachment is configured to at least partially cover the barrel flange and surround a proximal barrel opening of the vacuum syringe, and the one or more keys protrude from the priming attachment and are configured to extend partially over the proximal opening.

18. The priming system of claim 16, wherein the priming attachment comprises one or more engagement features configured to snap onto the barrel flange.

19. The priming system of claim 16, wherein each of the one or more keys protrudes radially inward, is at least as deep as each of the one or more notches, and is wider than each of the one or more notches.

20. The priming system of claim 16, wherein the one or more notches are located at different positions along a length of the plunger, wherein the different positions along the length of the plunger are configured to correspond with different volumes of fluid inside a barrel of the vacuum syringe.
